(19) United States
(12) Patent Application Publication
(10) Pub. No.: US 2022/0235284 A1
(43) Pub. Date: Jul. 28, 2022

(54) DECREASING REFINERY FOULING AND CATALYST DEACTIVATION

(71) Applicant: PHILLIPS 66 COMPANY, HOUSTON, TX (US)

(72) Inventors: Jinfeng Lai, Bartlesville, OK (US); Keith Lawson, Bartlesville, OK (US)

(73) Assignee: PHILLIPS 66 COMPANY, HOUSTON, TX (US)

(21) Appl. No.: 17/586,150

(22) Filed: Jan. 27, 2022

Related U.S. Application Data
(60) Provisional application No. 63/142,192, filed on Jan. 27, 2021.
(60) Provisional application No. 63/142,208, filed on Jan. 27, 2021.

(51) Int. Cl. C10G 75/00; G01N 24/08; G01N 33/28

(52) U.S. Cl. CPC C10G 75/00; G01N 24/085; C10G 2300/70; C10G 2300/4075; G01N 33/2835

(57) ABSTRACT

Processes for preventing or minimizing the rate of upgrading catalyst deactivation in a petroleum refinery, preventing or minimizing the rate of silicone-containing deposits within refinery process equipment, or both utilizing high-field proton nuclear magnetic spectroscopy (NMR) to rapidly measure concentrations of polydimethylsiloxanes (PDMS) and its thermal degradation products in potential refinery feed stock and refinery intermediate streams with high sensitivity and precision.

### CROSS-REFERENCE TO RELATED APPLICATIONS

This application is a non-provisional application which claims the benefit of and priority to U.S. Provisional Application Ser. Nos. 63/142,192 and 63/142,208 filed Jan. 27, 2021, entitled “Decreasing Refinery Fouling and Catalyst Deactivation,” both of which are hereby incorporated by reference in its entirety.

### STATEMENT REGARDING FEDERALLY SPONSORED RESEARCH OR DEVELOPMENT

None.

### FIELD OF THE INVENTION

Processes for preventing or minimizing poisoning of refinery catalysts and fouling of refinery process equipment by using high-field proton nuclear magnetic spectroscopy (NMR) to rapidly measure concentrations of polydimethylsiloxanes (PDMS) and PDMS thermal degradation products in a feedstock comprising unrefined petroleum and refinery intermediate streams with high sensitivity and precision.

### BACKGROUND

Polydimethylsiloxane (PDMS) is typically used as an anti-foaming agent during deep water crude oil recovery. However, this siloxane polymer (or thermal degradation products of PDMS) thermally decomposes at the high temperatures utilized in crude oil refineries, including, but not limited to, FCC and hydrocrackers, delayed cokers, refinery hydrotreaters and refinery process heaters. The compounds that are produced during PDMS thermal degradation can poison catalysts used in various processes, such as hydrotreaters and reformers, and can cause fouling of refinery process equipment by increasing the deposition rate of solid compounds (such as, but not limited to, silicon oxycarbide) inside refinery conduits and process furnaces.

Further, in certain petroleum refineries that comprise a delayed coker, PDMS is sometimes added directly into the coke drum to decrease foaming as the coker thermally cracks the feed and vapors emerge. PDMS in the crude oil feed is likely an even larger contributor to silicon poisoning of refining catalysts than the PDMS added to the delayed coker, probably due to it being exposed to high temperature for a longer period than the PDMS added in the coker.

Some conventional methods employed to measure silicon in crude oils comprise elemental analysis by inductively coupled plasma-atomic emission spectrometry (ICP-AES). However, such methods only provide total silicon content, do not distinguish inorganic silicon from organic silicon and are not specific for PDMS. Other methods measure PDMS content include spectroscopic methods, such as Fourier-transform infrared spectroscopy (FTIR) and Raman spectrometry. However, these methods are lack adequate sensitivity to detect concentrations of PDMS that are found in crude oils (typically, only a few parts per million [ppm]). What is needed is are fast and accurate methods that can rapidly and accurately measure low concentrations of both PDMS and its thermal degradation products in crude oils to prevent (or reduce the rate of) catalyst deactivation and refinery equipment fouling due to PDMS.

### BRIEF SUMMARY OF THE DISCLOSURE

The present inventive disclosure relates to methods for decreasing the rate of refinery catalyst deactivation and/or fouling of petroleum refinery equipment, comprising: a) obtaining a liquid sample from a feedstock comprising unrefined petroleum and diluting the liquid sample in a nuclear magnetic resonance spectroscopy (NMR) solvent that is fully miscible with the liquid sample to produce a diluted sample; b) adding a known amount of an internal control comprising a compound that contains at least one siloxane group to the diluted sample to produce an NMR sample; c) performing high-field proton NMR spectroscopy on the NMR sample to produce an NMR signal comprising free induction decay; d) detecting the NMR signal and performing Fourier transformation on the NMR signal to produce NMR spectral data; e) calculating the concentration of PDMS in the liquid sample by integrating a first peak present in the NMR spectral data located at 0.09 ppm proton chemical shift to produce PDMS peak area data and integrating a second peak present in the NMR spectral data that corresponds to the internal control to produce internal control peak area data, and calculating a PDMS concentration in the liquid sample using the PDMS peak area data and the internal control peak area data; f) mixing the feedstock with at least one additional feedstock comprising unrefined petroleum to produce a refinery feedstock mixture when the calculated PDMS concentration in the liquid sample is below a defined threshold concentration, wherein the at least one additional feedstock comprises a concentration of PDMS that is less than the threshold concentration and wherein the refinery feedstock mixture comprises a concentration of PDMS that is less than the threshold concentration; g) refining the refinery feedstock mixture.

In some embodiments, refining a feedstock comprising unrefined petroleum that comprises a concentration of PDMS that is at or above the threshold concentration causes at least one effect selected from: decreasing the catalytic activity of one or more refinery process catalysts by at least five percent and increasing the rate of fouling within refinery furnaces and piping by at least five percent.

In some embodiments, the second peak is located at 0.065 ppm 1H chemical shift in the NMR spectral data and corresponds to an internal control comprising hexamethyldisiloxane. In some embodiments, the internal control comprising hexamethyldisiloxane is diluted to a final concentration in the sample that is between 1 and 50 ppm.

In some embodiments, the NMR spectroscopy solvent comprises deuterated chloroform.

In some embodiments, the high-field proton NMR spectroscopy is performed at a pulse frequency of at least 300 MHz. In some embodiments, the detecting is performed by a digital quadrature detection receiver that includes at least one integrated digitizer.

In some embodiments, part f) of the process comprises rejecting the feedstock comprising unrefined petroleum as a petroleum refinery feedstock when the calculated PDMS concentration in the liquid sample is at or above a defined threshold concentration, wherein refining a refinery feedstock containing a concentration of PDMS that is at or above the threshold concentration causes at least one of: a decrease in catalytic lifespan for one or more refinery process catalysts and an increased rate of silicon-containing deposit formation within refinery process equipment. In some embodiments, the threshold concentration is at least 3 ppm.

In some embodiments, a concentration of PDMS that is at or above the threshold concentration results in at least one of: at least a 1 percent decrease in catalytic lifespan for one or more refinery upgrading catalysts and at least a 1 percent increased rate of silicon-containing deposit formation within refinery process equipment.

Certain embodiments comprise a method for scheduling the maintenance of petroleum refinery equipment and catalysts by measuring the concentration of polydimethylsiloxane (PDMS), comprising: a) obtaining a liquid sample from a feedstock comprising unrefined petroleum and diluting the liquid sample in a nuclear magnetic resonance spectroscopy (NMR) solvent that is fully miscible with the liquid sample to produce a diluted sample; b) adding a known amount of an internal control comprising a compound that contains at least one siloxane group to the diluted sample to produce an NMR sample; c) performing high-field proton NMR spectroscopy on the NMR sample to produce an NMR signal comprising free induction decay; d) detecting the NMR signal and performing Fourier transformation on the NMR signal to produce NMR spectral data; e) calculating the concentration of PDMS in the liquid sample by integrating a first peak present at in the NMR spectral data located at 0.09 ppm proton NMR chemical shift to produce PDMS peak area data and integrating a second peak present at in the NMR spectral data that corresponds to the internal control to produce internal control peak area data, and calculating a PDMS concentration in the liquid sample using the PDMS peak area data and the internal control peak area data; f) upgrading the feedstock comprising unrefined petroleum in a petroleum refinery, wherein the calculated PDMS concentration in the liquid sample is utilized to determine the time interval between refinery maintenance procedures comprising at least one of: cleaning silicon-containing deposits from refinery equipment, replacing refinery process catalysts and regenerating refinery process catalysts.

In some embodiments, the time interval that is determined in part f) minimizes refinery operational capital expenditures while maximizing the time interval between refinery maintenance procedures.

In some embodiments, the second peak is located at 0.065 ppm proton NMR chemical shift in the NMR spectral data and corresponds to an internal control comprising hexamethyldisiloxane. In some embodiments, the internal control comprising hexamethyldisiloxane is diluted to a final concentration in the sample that is between 1 and 50 ppm.

In some embodiments, the nuclear magnetic resonance spectroscopy solvent comprises deuterated chloroform.

In some embodiments, the high-field proton NMR spectroscopy is performed at a pulse frequency of at least 300 MHz. The method of claim 12, wherein the detecting is performed by a digital quadrature detection receiver that includes at least one integrated digitizer.

### BRIEF DESCRIPTION OF THE DRAWINGS

A more complete understanding of the present invention and benefits thereof may be acquired by referring to the follow description taken in conjunction with the accompanying drawings in which:

FIG. 1 is a graphical representation of proton (¹H) NMR spectral data obtained using the inventive methods described herein. Panel A depicts an entire proton (¹H) NMR spectrum obtained from a crude oil sample, while panel B depicts a magnified subset of the data depicted in panel A) showing well-defined peaks corresponding to PDMS and internal control hexamethyldisiloxane (HMDSO).

FIG. 2 is a graphical representation of proton (¹H) NMR spectral data obtained using the inventive methods described herein, showing the value of diluting the HMDSO internal control to prevent peak interference.

FIG. 3 is a graphical representation of proton (¹H) NMR spectral data obtained using the inventive methods described herein, showing discrete proton (¹H) NMR peaks obtained for three known thermal degradation products of PDMS.

FIG. 4 is a graphical representation of proton (¹H) NMR spectral data obtained using the inventive methods described herein, showing discrete proton (¹H) NMR peaks obtained for three known thermal degradation products of PDMS.

The invention is susceptible to various modifications and alternative forms, specific embodiments thereof are shown by way of example in the drawings. The drawings may not be to scale. It should be understood that the drawings are not intended to limit the scope of the invention to the particular embodiment illustrated.

### DETAILED DESCRIPTION

The present disclosure provides processes to describes a high-field nuclear magnetic resonance (NMR) measurement of trace levels of polydimethylsiloxanes (PDMS) in crude oils, which allows rapid assessment of candidate crudes before being processed in a commercial refinery. The use of high-field NMR coupled with high dynamic range receiver enables the detection of PDMS at the low parts-per-million level that is sufficient to cause catalyst deactivation in processing units, such as hydrotreaters and reformers. A second embodiment allows precise quantitation of known PDMS degradation products that are formed during thermal degradation of PDMS in various refinery processes. These methods enable the measurement of PDMS and PDMS thermal degradation products at concentrations of less than 10 part-per-million (ppm) (optionally, less than 5 ppm; optionally, as low as 1 ppm), which can be detrimental to the refining process. The method has the advantages of efficiency, lower detection limit, great accuracy, and better precision than prior methods for measuring PDMS and its thermal degradation products. In addition, the method is specific for PDMS content (and PDMS thermal degradation product content) instead of merely total silicon content.

The processes described can rapidly detect levels of PDMS in crude oil samples, and specific PDMS thermal degradation products created during different refining processes. This knowledge can decrease the rate of deactivation of refinery process catalysts by PDMS-contaminated crude oils by either preventing utilization of a PDMS-containing crude oil as refinery feedstock when that crude oil comprises a concentration of PDMS that is above a given threshold concentration. An alternative embodiment provides for preemptive dilution of above-threshold PDMS-containing feedstocks comprising unrefined petroleum with at least one additional crude oil feedstock containing a concentration of PDMS that is less than the threshold concentration, until the overall concentration of PDMS in the crude oil feedstock is less than the threshold concentration.

In some embodiments, quantitation of PDMS levels (and PDMS thermal degradation products) at the single-digit ppm level can better inform the proper time intervals for conducting refinery maintenance by allowing calculation of expected decreases in catalyst activity over time due to deactivation by PDMS and/or PDMS thermal degradation products present in the feedstock or refinery intermediate stream. In addition, quantitation of the concentration of PDMS (and PDMS thermal degradation products) can provide information, that when combined with historical knowledge regarding the rate which silicon-containing deposits form inside refinery process equipment due to the presence of PDMS and/or PDMS thermal degradation products in the feedstock or refinery intermediate stream. This, in turn, informs the most appropriate time interval (that minimizes operational cost and shutdown-time while maximizing operational profit) between refinery maintenance procedures that may include at least one of: cleaning silicon-containing deposits from refinery equipment, replacing refinery process catalysts and regenerating refinery process catalysts.

Determination of the threshold concentration for PDMS in a given potential refinery feedstock comprising crude oil may depend on a number of variables that include (but are not limited to) availability of alternative crude oil feedstock, types of catalysts utilized in one or more refinery processes, inclusion of a delayed coker in the refining process, expected time interval until the next planned refinery maintenance shutdown (i.e., turnaround), etc. In certain embodiments, the threshold value may comprise a PDMS concentration in the range from 1-100 ppm; alternatively, a PDMS concentration in the range from 1-50 ppm; alternatively, a PDMS concentration in the range from 1-20 ppm; alternatively, a PDMS concentration in the range from 1-10 ppm; alternatively, a PDMS concentration in the range from 1-5 ppm; alternatively, a PDMS concentration of 50 ppm or more; alternatively, a PDMS concentration of 20 ppm or more; alternatively, a PDMS concentration of 10 ppm or more; alternatively, a PDMS concentration of 5 ppm or more; alternatively, a PDMS concentration of 3 ppm or more; alternatively, a PDMS concentration of 2 ppm or more.

Crude oil contains a vast multitude of complex molecules. All hydrocarbon molecules (and any other molecules containing a hydrogen atom) show proton (¹H) NMR resonance signals, leading to complex NMR spectra. Thus, development of the present inventive processes required extensive knowledge of both NMR technology and crude oil chemistry to precisely quantitate the extremely small (¹H) NMR peak associated with PDMS in the low (single digit) ppm range within the complex matrix of a crude oil sample (extraction of PDMS into a solvent is avoided). The resulting process can quantify PDMS within a crude oil sample faster, with greater sensitivity, accuracy and precision than conventional methods.

One of the novelties of the method is the utilization of proton (¹H) NMR instead of silicon (²⁹Si) NMR to detect PDMS. The this greatly decreases the time needed to accurately measure the concentration and greatly increases sensitivity of the process down to the low ppm range for both PDMS and its thermal degradation products. In fact, the sensitivity of proton (¹H) NMR is approximately 2,700-fold higher than ²⁹Si NMR. Secondly, the T1 spin-lattice relaxation time of proton (¹H) NMR is about ten times shorter than silicon NMR. Because of this, proton (¹H) NMR signals sufficient to accurately quantify low ppm concentrations of PDMS are acquired much more quickly than if the process utilized ²⁹Si NMR. For the present processes, total acquisition time is less than 20 minutes, whereas ²⁹Si NMR can require multiple days to acquire a sufficient PDMS signal to measure the low levels of PDMS that are quantitated by the present inventive methods.

An additional advantage of the present inventive methods is the inclusion of a highly diluted internal standard comprising hexamethyldisiloxane (HMDSO), which allows precise quantification of PDMS (and/or its thermal degradation products) at low ppm sensitivity. HMDSO contains a siloxane group and thus has a similar basic structure to PDMS. Under the conditions associated with the inventive processes, HMDSO produces a distinct singlet resonance that is well-isolated from other NMR signals associated with PDMS or compounds in the crude oil. This eliminates any matrix interference effects and allows accurate quantitation of PDMS by integration of relative peak area compared to the HMDSO control. The present methods utilize far less HMDSO internal standard than is typically used in conventional NMR methods. Typically, 110 ul of a 100 ppm HDMSO standard solution is added to each 0.5 g crude sample prior to analysis for a final sample PDMS concentration of about 20 ppm. Extensive pre-dilution of the internal standard to 100 ppm allows full resolution of PDMS signals from those of the internal standard, which gives more accurate quantitation. An additional benefit of the process is that by utilizing an internal standard, no calibration calculations are needed, which reduces total analysis time and determination error. Further, full isolation of the NMR signals for the internal standard, (HMDSO) the PDMS, and three thermal degradation products of PDMS allows easy integration of the full peak for each, and consequently, more accurate quantitation of PDMS and its degradation products.

The use of high-field NMR coupled with high dynamic range receiver enables the detection of PDMS at parts-per-million level which causes the catalyst deactivation in processing units, such as hydrotreaters and reformers. Two distinct embodiments are able to generate different information of value in regulating the operation of a commercial crude oil refinery. The first embodiment quantifies the concentration of PDMS in different potential crude feedstocks, allowing either selection of only feed stocks that do not contain sufficient PDMS to detrimentally affect refinery operation, or allowing a an accurate estimate of how refining a given PDMS containing crude oil can be expected to detrimentally affect refinery operations, including a determination of expected catalyst lifetime and the rate of silicon deposits inside refining process equipment.

A second, distinct embodiment allows accurate detection and quantitation of specific PDMS thermal degradation products that are present in refinery intermediate streams. The embodiment has high sensitivity (i.e., low single-digit ppm level) and precision, and allows the accurate prediction of the deposition rate for these PDMS degradation products inside refinery process equipment (e.g., delayed coking heaters, hydrotreaters, etc.). The rationale behind this embodiment is that PDMS is known to thermally decompose into smaller organosilicon compounds during refining of a crude oil in a commercial oil refinery, and particularly within the high heat of a delayed coker apparatus that thermally cracks a residual oil (typically derived from a vacuum distillation apparatus) comprising high molecular weight hydrocarbons into lighter products that are fractionated into intermediate products (e.g., naphtha, light and heavy gas oils, etc.) that can be redirected for upgrading by other refining processes. Such processing units are well-understood in refining field; thus, further description is outside of the scope of this disclosure.

Understanding how to maximize time interval between required refinery unit maintenance and/or catalyst regeneration and/or replacement can dramatically improve efficiency, and more efficient refinery operation leads to increased profit. For example, levels of silicon beyond 2 ppm in a naphtha feed stream to a hydrotreater can often cause severe hydrotreating catalyst deactivation through decreased surface area, pore volume and blocking of the catalyst active sites. Levels of silicon in a reformer feed stream that exceed 0.5 ppm result in significant deactivation of reformer catalysts through metal agglomeration and loss of chloride ions from the catalyst active sites.

Often, PDMS is added to the delayed coker feed stream in order to decrease foaming inside the delayed coker unit. PDMS is a type of polymer and its molecular weight varies from 10K to 200K Dalton depending on the manufacturer and batch. Typically, approximately 50% to 90% of PDMS that is present in the feed stream to a delayed coker decomposes into one or more cyclic siloxane degradation products (for example, see Table 1: D3, D4, D5) inside the delayed coker. Any PDMS that does not degrade has a boiling point that is higher than the temperature that is maintained within the coker (generally, approximately 454° C.). Thus, any undegraded PDMS remains in the coker liquid and does not transfer into the coker vapors that migrate out of the coker drum and are received by the coker fractionator. The selectivity toward production of each of these cyclic siloxane degradation products is often similar, but the total quantity of thermal degradation products produced depends on the quantity of PDMS that is added to the crude oil feed (or intermediate feed to the delayed coker), the molecular weight (or viscosity) of the PDMS, and the coking temperature.

Three common PDMS thermal decomposition products are shown in Table 1. Each product is a monocyclic siloxane that is characterized by a different boiling point from the others (see Table 1). Thus, following fractionation by boiling point in the fractionator of a delayed coker, each of the products listed in Table 1 is often directed to one or more distinct refinery upgrading processes. As shown in Table 1, D3 (hexamethylcyclotrisiloxane) is typically directed to refinery upgrading processes that produce gasoline, D4 (octamethylcyclotetrasiloxane) may be directed to either gasoline or diesel upgrading pathways depending on the cut point of the coker fractionator, and D5 (decamethylcyclopentasiloxane) is typically directed to refinery upgrading processes that produce diesel fuel. Often, the refinery upgrading processes mentioned above comprise hydrotreating the various fractions obtained from the coker fractionator. The hydrotreating catalysts utilized are known to be sensitive to deactivation by contact with the thermal degradation products shown in Table 1. Thus, accurate quantitation of the concentration of one or more of D3, D4 and D5 thermal degradation products in a given refinery intermediate product stream (such as, but not limited to, a naphtha or gasoil fraction obtained from a delayed coker fractionator) can provide important information regarding the expected catalytic lifespan of one or more refinery process catalysts that facilitate upgrading that fraction to a transportation fuel, and/or the expected rate of deposition of solids inside refinery process equipment. This, in turn, informs a determination of the maximum (or most efficient) time interval refinery between performing refinery maintenance procedures comprising at least one of: cleaning silicon-containing deposits from refinery equipment, replacing refinery process catalysts and regenerating refinery process catalysts. In this context, most efficient refers to the time interval that best balances refinery process efficiency with the costs to perform periodic maintenance, thereby maximizing overall profit.

TABLE 1
Three thermal degradation products of PDMS that are quantitated by the present methods.

| Cyclic | | | Boiling | |
|---|---|---|---|---|
| Siloxanes | Chemical Name | Structure | Point | Finished Product |
| D3 | Hexamethylcyclotrisiloxane | 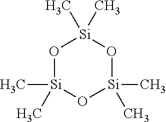 | 147° C. | Gasoline |
| D4 | Octamethylcyclotetrasiloxane | 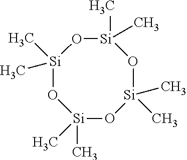 | 348° C. | Gasoline/Diesel |
| D5 | Decamethylcyclopentasiloxane | 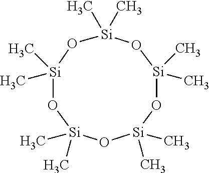 | 410° C. | Diesel |

Because each thermal degradation product (D3-D5) listed in Table 1 may be directed to one or more different catalytic upgrading process, the knowledge of the concentration of each PDMS thermal degradation product in a given refinery intermediate stream (such as, but not limited to, a fraction from a delayed coking unit fractionator, a hydrotreater feed, a reformer feed, coker naphtha, coker distillate, coker light gas oil and coker heavy gasoil) can assist in accurately estimating the rate at which upgrading catalysts in each upgrading pathway will be deactivated or poisoned due to presence of PDMS in the given refinery intermediate stream. In alternative embodiments, the concentration of each PDMS thermal degradation product can assist in accurately estimating whether solid silicon-containing deposits should be expected inside conduits and heaters for a given refinery process, and if so, the rate of accumulation of these deleterious deposits. Therefore, accurate quantitation of PDMS thermal degradation products provides valuable information to predict catalyst run lengths (i.e., lifespan) and maximize refinery process efficiency by avoiding premature refinery shutdown to replace these upgrading catalysts, remove deposits from process furnaces and conduits, or both. In certain embodiments, a threshold concentration serves as an indicator of an unrefined crude oil that may increase the rate silicone-containing solids deposition (fouling) and/or decrease the catalytic lifespan of one or more upgrading catalysts in the refinery to a degree that is commercially unacceptable. In some embodiments, the threshold concentration of PDMS or a thermal degradation product thereof may represent the concentration that is known to result in an increased rate of catalyst deactivation and/or silicone-containing deposit formation. Optionally, the threshold concentration may increase the rate of catalyst deactivation and/or silicone-containing deposit formation by at least 1%; alternatively, at least 2%; alternatively, at least 5%; alternatively, at least 10%; alternatively, at least 15%; alternatively, at least 20%; alternatively, at least 25%; alternatively, at least 50%.

The processes and systems disclosed herein provide numerous distinct advantages over conventional assays that attempt to quantify PDMS or total silicon content. One of the many advantages is that the present inventive processes can be applied to any type of crude oil regardless of viscosity (or any distillation fraction of a crude oil), as the crude sample is dissolved in a quantity of solvent and homogenized. This is faster and more sensitive than attempting to extract PDMS from a crude sample, then measuring PDMS in an only portion of the extraction solvent.

An additional advantage of the present processes and systems is a much lower detection limit than conventional methods, with greater accuracy and far better precision. It can accurately determine the PDMS content in a crude sample down to just a few parts per million (ppm). Further, the process is highly specific for PDMS and certain of its thermal degradation products. This is important for predicting the rate of catalyst silicon poisoning in refinery processes where the PDMS contaminated crude is utilized as feed stock. Plans for replenishing or regenerating various refinery catalysts can be more efficiently planned and executed based on this knowledge.

An additional advantage is the utilization of an easily identifiable, diluted internal standard, which is distinct from the proton NMR peaks of PDMS and its thermal degradation products, thereby allowing accurate determination of the concentration of PDMS and its degradation products by integration of distinct NMR peaks. This not only reduces total time required to make the measurement, but also increases accuracy and precision in the measurement.

### EXAMPLES

The following examples are representative of one embodiment of the inventive processes and systems disclosed herein, and the scope of the invention is not intended to be limited to the embodiment specifically disclosed. Rather, the scope is intended to be as broad as is supported by the claims listed below.

### Example 1

A sample containing 0.5 gram of crude petroleum oil was dissolved in 1.25 gram of deuterated chloroform (NMR solvent) and 110 μl (approximately two drops) of an internal standard comprising a 100 ppm solution of HMDSO in toluene. Use of deuterated chloroform as NMR solvent was chosen because it is fully miscible with crude oil, thereby avoiding any need to extract PDMS (or degradation products) from the crude oil sample and assuring that all the PDMS in the sample is analyzed. The sample is then homogenized prior to subjecting it to proton NMR analysis.

A high-field NMR device was coupled with a high dynamic range receiver and amplifier as follows: Magnet: Bruker Ascend™ 400 MHz (9.4 telsa) high field NMR magnet Console: Bruker AVANCE™ III HD 400 MHz high performance digital NMR console. Receiver: Enhanced 2G Digital Quadrature Detection Receiver (RXAD/2) with integrated high-performance ADCs (analog-to-digital converter, or digitizer). This receiver provides the highest dynamic range, high digital resolution and large bandwidth digital filtering. Amplifier: Bruker BLAXH500/100 amplifier, 20-100 Watt linear excitation pulse power for ¹H channel. Nuclear Channel: Proton (¹H) channel Pulse: 45 degree pulse Scan: 128 scans.

In some embodiments of the present inventive processes, the high-field NMR instrument utilized has a processing frequency of at least 300 MHz. In certain experiments, the instrument utilized has a processing frequency of at least 400 MHz. It is clear that higher frequency NMR instruments (e.g., 500 MHz, 600 MHz, etc.) would also be suitable for use with the present inventive methods.

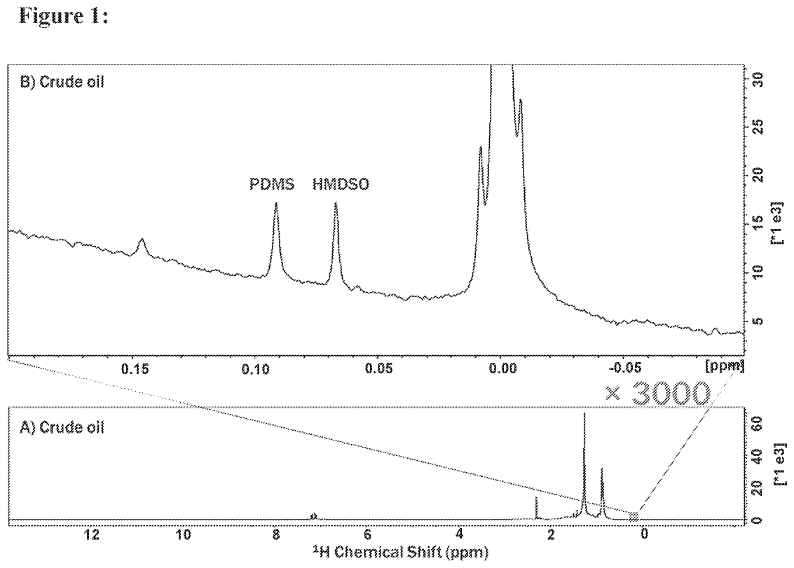

The resulting NMR spectra for a crude oil sample containing PDMS and the HDMSO internal standard is shown in FIG. 1. The bottom frame of FIG. 1 shows the full NMR spectral data (comprising a Fourier transformation of free induction decay [FID]) obtained from using 21 W power to generate an excitation radiofrequency pulse of 25 kHz that excited the sample. In certain embodiments, the power used to create the NMR radiofrequency pulse may be at least 10 W; optionally, at least 20 W; optionally, at least 30 W; optionally, at least 40 W. The small highlighted region in the lower panel is magnified in the upper panel to clearly show adjacent NMR peaks for both PDMS (0.09 ppm ¹H chemical shift) and the HMDSO internal sample (0.065 ppm ¹H chemical shift). It is clear from the figure that that the peaks for both HMDSO and PDMS are both extremely small relative to many other compounds in the sample. Yet, the peaks for both HMDSO and PDMS are both well-isolated and can be easily quantified by integration using commercially-available software.

### Example 2

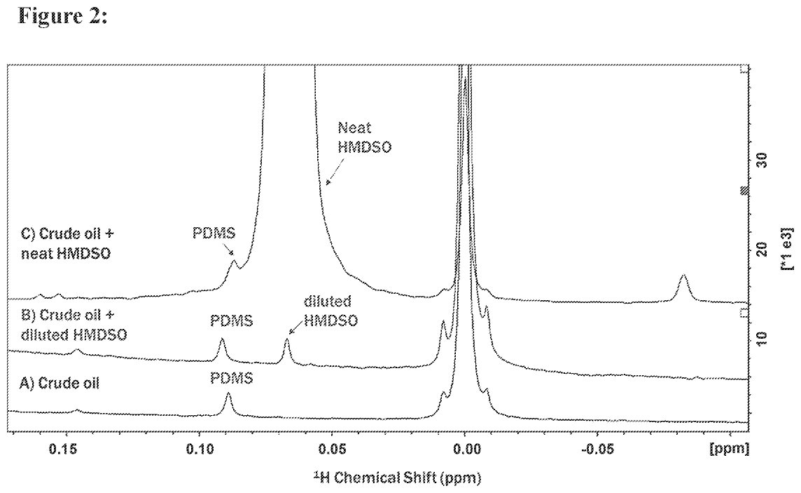

This example shows the advantage of diluting the internal standard to a final concentration in the crude oil sample that is in the range from 1 to 50 ppm prior to NMR analysis. Using the same apparatus and settings, 0.5 g of crude oil samples (900 mg/ml density @ 20° C.=555 μl) were prepared that contained a) Crude oil+PDMS, no HMDSO; b) Crude oil+PDMS+110 ul of HMDSO diluted to 100 ppm, and c) Crude oil+PDMS+110 ul of neat (undiluted) HMDSO. Results are shown in the multiple NMR spectra presented in FIG. 2. Utilizing HMDSO standard diluted to a final concentration of 16.7 ppm in the crude oil sample ensures the complete resolution of adjacent PDMS and HMDSO peaks, which allows accurate integration of the PDMS and HMDSO peak areas and and accurate measure of the PDMS concentration relative to the known HMDSO concentration. In certain embodiments, the concentration of the HMDSO ranges from 1 to 50 ppm; optionally, the concentration of the HMDSO ranges from 5 to 30 ppm; optionally, the concentration of the HMDSO ranges from 5 to 20 ppm. FIG. 2 clearly demonstrates that utilizing an undiluted (neat) HMDSO internal standard interferes with the adjacent PDMS peak, preventing accurate resolution and quantitation of the range of PDMS concentrations (e.g. 1-10 ppm) that are typically found in crude oil samples.

### Example 3

This example demonstrates the resolution and quantitation of three different thermal degradation products of PDMS (i.e., D3, D4, D5) that are produced during the refining of PDMS-contaminated crude oil (as outlined above). Using the same apparatus and equipment settings as in Example 1, a sample of a coker liquid refinery stream (i.e., coker effluent) was analyzed by proton (¹H) NMR. The delayed coker liquid effluent was the liquid effluent from a delayed coker that had processed a PDMS-containing coker feed (e.g., FCC slurry, vacuum residuum, etc.). The high temperature within the delayed coker led to the thermal degradation of PDMS in the delayed coker, which produced PDMS thermal degradation products that exited the delayed coker as vapors and remained in the delayed coker liquid effluent.

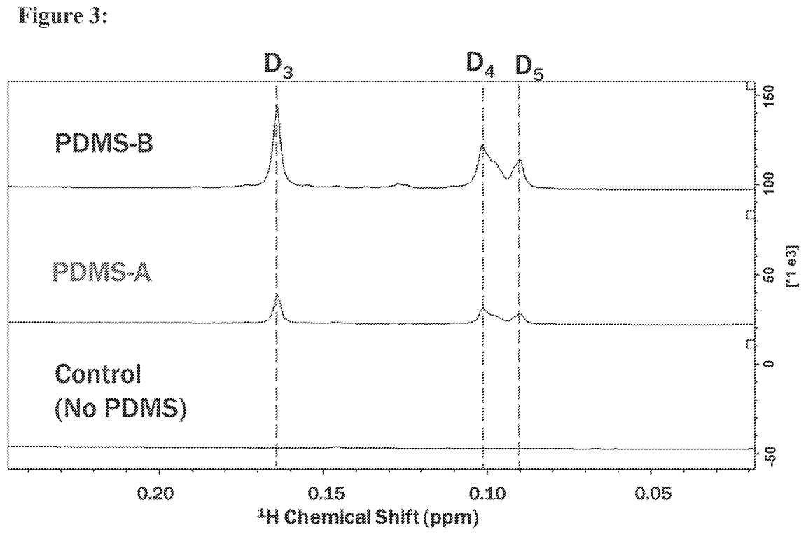

Results of the NMR analysis are shown in the stacked NMR spectra presented in FIG. 3. The Control (bottom) represent a small, magnified region (3000×) within a full NMR spectrum obtained from a delayed coker fluid (i.e., coker effluent) derived from the processing of a crude oil that contained no PDMS. The PDMS-A and PDMS-B samples (middle and top spectrums) represent a small region (3000× Zoom) within a full NMR spectra obtained from two coker fluids samples that were derived from the processing of crude oils containing residual levels of PDMS contamination (<10 ppm).

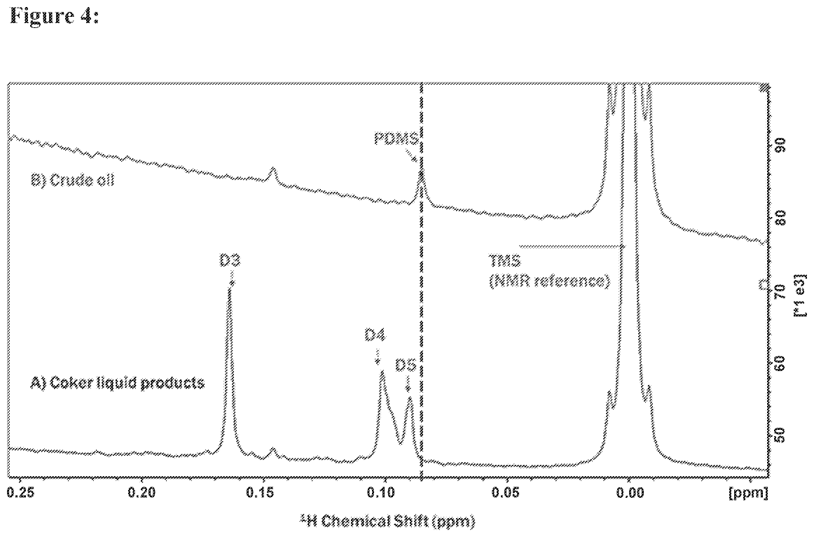

FIG. 4 shows that NMR peaks for all three degradation products (D3, D4, and D5) can be clearly resolved at 0.165 ppm proton NMR chemical shift corresponding to hexamethylcyclotrisiloxane (D3), at 0.10 ppm proton (¹H) NMR chemical shift corresponding to octamethylcyclotetrasiloxane (D4) and at 0.09 ppm proton NMR chemical shift corresponding to decamethylcyclopentasiloxane (D5). These peaks were clearly distinguishable from the peak at 0.085 corresponding to PDMS and were quantitated using the inventive process as described. It is important to note that the boiling point of PDMS is higher than the typical operating temperature of a delayed coker. For this reason, undegraded PDMS does not leave the delayed coker drum intact, and instead remains with the solidified coke that forms inside each coker drum. Typically, only PDMS thermal degredations products leave the delayed coker as part of the coker fluid effluent.

The three thermal degradation products (D3-D5) have different boiling points (see Table 1). Thus, when fractionated by a delayed coker fractionator, each thermal degradation product predominantly segregates with a distinct fraction that is derived from a coker fractionator that may include (but is not limited to) coker naphtha, coker distillate, coker light gas oil and coker heavy gas oil. These fractions are typically each directed to a distinct upgrading pathway in the refinery (e.g., reformers and hydrotreaters) (see Table 1, last column) to produce blend stocks for different finished transportation fuel products (e.g., gasoline and diesel). Thus, knowledge of the concentration of each PDMS thermal degradation product in a given fraction can better inform the refining process, including expected catalyst lifetime in refinery process units, thereby allowing efficient scheduling of the best balance between refinery run length between turnarounds (i.e. the longest time period between replenishment or regeneration of refinery process catalysts, or cleaning of fouled refining process equipment such as heaters, conduits, etc.) before the efficiency of one or more catalytic upgrading process decreases beyond an acceptable level due to the deleterious effects of PDMS.

In the present disclosure, the term “crude oil” is synonymous with crude petroleum that has not been processed in a petroleum refinery. The origin of the petroleum is not of significance to the operability of the process.

Although the systems and processes described herein have been described in detail, it is understood that various changes, substitutions, and alterations can be made without departing from the spirit and scope of the invention as defined by the following claims.

We claim:

1. A method for decreasing the rate of fouling of catalyst deactivation and/or petroleum refinery equipment, comprising:
a) obtaining a liquid sample from a feedstock comprising unrefined petroleum and diluting the liquid sample in a nuclear magnetic resonance spectroscopy (NMR) solvent that is fully miscible with the liquid sample to produce a diluted sample;
b) adding a known amount of an internal control comprising a compound that contains at least one siloxane group to the diluted sample to produce an NMR sample;
c) performing high-field proton NMR spectroscopy on the NMR sample to produce an NMR signal comprising free induction decay;
d) detecting the NMR signal and performing Fourier transformation on the NMR signal to produce NMR spectral data;
e) calculating the concentration of PDMS in the liquid sample by integrating a first peak present in the NMR spectral data located at 0.09 ppm proton chemical shift to produce PDMS peak area data and integrating a second peak present in the NMR spectral data that corresponds to the internal control to produce internal control peak area data, and calculating a PDMS concentration in the liquid sample using the PDMS peak area data and the internal control peak area data;
f) mixing the feedstock with at least one additional feedstock comprising unrefined petroleum to produce a refinery feedstock mixture when the calculated PDMS concentration in the liquid sample is below a defined threshold concentration, wherein the at least one additional feedstock comprises a concentration of PDMS that is less than the threshold concentration and wherein the refinery feedstock mixture comprises a concentration of PDMS that is less than the threshold concentration;
g) refining the refinery feedstock mixture.

2. The method of claim 1, wherein refining a feedstock comprising unrefined petroleum that comprises a concentration of PDMS that is at or above the threshold concentration causes at least one effect selected from: decreasing the catalytic activity of one or more refinery process catalysts by at least five percent and increasing the rate of fouling within refinery furnaces and piping by at least five percent.

3. The method of claim 1, wherein the second peak is located at 0.065 ppm ¹H chemical shift in the NMR spectral data and corresponds to an internal control comprising hexamethyldisiloxane.

4. The method of claim 3, wherein the internal control comprising hexamethyldisiloxane is diluted to a final concentration in the sample that is between 1 and 50 ppm.

5. The method of claim 1, wherein the NMR spectroscopy solvent comprises deuterated chloroform.

6. The method of claim 1, wherein the high-field proton NMR spectroscopy is performed at a processing frequency of at least 300 MHz.

7. The method of claim 1, wherein the detecting is performed by a digital quadrature detection receiver that includes at least one integrated digitizer.

8. The method of claim 1, wherein part f) comprises rejecting the feedstock comprising unrefined petroleum as a petroleum refinery feedstock when the calculated PDMS concentration in the liquid sample is at or above a defined threshold concentration, wherein refining a refinery feedstock containing a concentration of PDMS that is at or above the threshold concentration causes at least one of: a decrease in catalytic lifespan for one or more refinery process catalysts and an increased rate of silicon-containing deposit formation within refinery process equipment.

9. The method of claim 1, wherein the threshold concentration is at least 3 ppm.

10. The method of claim 1, wherein the threshold concentration of PDMS results in at least one of: at least a 1 percent decrease in catalytic lifespan for one or more refinery upgrading catalysts and at least a 1 percent increased rate of silicon-containing deposit formation within refinery process equipment.

11. A method for improving the maintenance schedule of petroleum refinery equipment and catalysts, comprising:
a) obtaining a liquid sample from a feedstock comprising unrefined petroleum and diluting the liquid sample in a nuclear magnetic resonance spectroscopy (NMR) solvent that is fully miscible with the liquid sample to produce a diluted sample;
b) adding a known amount of an internal control comprising a compound that contains at least one siloxane group to the diluted sample to produce an NMR sample;
c) performing high-field proton NMR spectroscopy on the NMR sample to produce an NMR signal comprising free induction decay;
d) detecting the NMR signal and performing Fourier transformation on the NMR signal to produce NMR spectral data;
e) calculating the concentration of PDMS in the liquid sample by integrating a first peak present at in the NMR spectral data located at 0.09 ppm proton NMR chemical shift to produce PDMS peak area data and integrating a second peak present at in the NMR spectral data that corresponds to the internal control to produce internal control peak area data, and calculating a PDMS concentration in the liquid sample using the PDMS peak area data and the internal control peak area data;
f) upgrading the feedstock comprising unrefined petroleum in a petroleum refinery, wherein the calculated PDMS concentration in the liquid sample is utilized to determine the time interval between refinery maintenance procedures comprising at least one of: cleaning silicon-containing deposits from refinery equipment, replacing refinery process catalysts and regenerating refinery process catalysts.

12. The method of claim 11, wherein part e) comprises calculating the concentration of at least one thermal degradation product of PDMS in the liquid sample by integrating at least one peak present in the NMR spectral data selected from a peak at 0.09 ppm proton NMR chemical shift corresponding to decamethylcyclopentasiloxane, a peak at 0.10 ppm proton NMR chemical shift corresponding to octamethylcyclotetrasiloxane and a peak at 0.165 ppm proton NMR chemical shift corresponding to hexamethylcyclotrisiloxane to produce PDMS degradation product peak area data, integrating a control peak present in the NMR spectral data that corresponds to the internal control to produce internal control peak area data, and calculating the concentration of at least one of the PDMS thermal degradation products in the liquid sample using the PDMS degradation product peak area data obtained from at least one PDMS thermal degradation product and the internal control peak area data.

13. The method of claim 11, wherein the time interval that is determined in part f) minimizes refinery operational capital expenditures while maximizing the time interval between refinery maintenance procedures.

14. The method of claim 11, wherein the refinery intermediate stream is a fraction derived from a coking unit fractionator selected that is selected from coker naphtha, coker distillate, coker light gas oil and coker heavy gasoil.

15. The method of claim 11, wherein the second peak is located at 0.065 ppm proton NMR chemical shift in the NMR spectral data and corresponds to an internal control comprising hexamethyldisiloxane.

16. The method of claim 15, wherein the internal control comprising hexamethyldisiloxane is diluted to a final concentration in the sample that is between 1 and 50 ppm.

17. The method of claim 11, wherein the nuclear magnetic resonance spectroscopy solvent comprises deuterated chloroform.

18. The method of claim 11, wherein the high-field proton NMR spectroscopy is performed at a processing frequency of at least 300 MHz.

19. The method of claim 11, wherein the detecting is performed by a digital quadrature detection receiver that includes at least one integrated digitizer.
